(19) United States
(12) Patent Application Publication
(10) Pub. No.: US 2018/0024137 A1
(43) Pub. Date: Jan. 25, 2018

(54) METHODS FOR COMPARING A STRUCTURE OF A FIRST BIOMOLECULE AND A SECOND BIOMOLECULE

(71) Applicant: Sarfaraz K. Niazi, Deerfield, IL (US)

(72) Inventor: Sarfaraz K. Niazi, Deerfield, IL (US)

(21) Appl. No.: 15/720,755

(22) Filed: Sep. 29, 2017

Related U.S. Application Data
(63) Continuation of application No. 13/961,571, filed on Aug. 7, 2013.
(60) Provisional application No. 61/830,889, filed on Jun. 4, 2013.

(51) Int. Cl. G01N 33/68; G01N 21/64

(52) U.S. Cl. CPC G01N 33/6803; G01N 21/6486

(57) ABSTRACT

The present disclosure provides methods to assess structural similarity of a first biomolecule and a second biomolecule by detecting one or more responses of the first and second biomolecule to thermodynamic stress conditions induced by osmotic and dielectric changes including, detecting a shift in fluorescence emission and/or a change in the intensity of the emission.

### CROSS REFERENCE TO RELATED APPLICATION

This application is a continuation of U.S. patent application Ser. No. 13/961,571, filed on Aug. 7, 2013 which claims the benefit of U.S. Patent Application Ser. No. 61/830,889, filed on Jun. 4, 2013, the contents of each of which are incorporated herein by reference in their entireties.

### FIELD

The disclosure relates to methods of evaluating the similarity (e.g., structural similarity) of therapeutic proteins, antibodies and peptides (each referred to herein as a biomolecule) to a second biomolecule (e.g., a reference biomolecule) to ensure comparable safety and efficacy.

### BACKGROUND

Biosimilars, also known as follow-on biologics, are biologic medical products whose active drug substance are made by a living organism or derived from a living organism by means of recombinant DNA or controlled gene expression methods.

Biosimilars and follow-on biologics are terms used to describe officially approved subsequent versions of innovator biopharmaceutical products made by a different sponsor following patent and exclusivity expiry on the innovator product. Biosimilars are also referred to as subsequent entry biologics (SEBs) in Canada. Reference to the innovator product is an integral component of the approval.

Unlike the more common small-molecule drugs, biologics generally exhibit high molecular complexity, and may be quite sensitive to changes in manufacturing processes. Biosimilar manufacturers do not have access to the innovator's molecular clone and original cell bank, nor to the exact fermentation and purification processes, nor to the active drug substance. They do have access to the commercialized innovator product and industry know-how. However, differences in impurities and/or breakdown products can have serious health implications. This has created a concern that copies of biologics might perform differently than the original branded version of the product. Consequently only a few subsequent versions of biologics have been authorized in the US through the simplified procedures allowed for small molecule generics, namely Menotropins (January 1997) and Enoxaparin (July 2010), and a further eight biologics through the 505(b)(2) pathway.

Biosimilars are subject to an approval process requiring substantial additional data to that required for chemical generics, although not as comprehensive as for the original biotech medicine. In order to be released to the public, biosimilars must be shown to be as close to identical to the parent biological product based on data compiled through clinical, animal and analytical studies. The results must demonstrate that they produce the same clinical results and are interchangeable with the referenced FDA licensed biological product already on the market. The US FDA has clearly enunciated the rules of the game and it is “on a product by product basis” and on the “totality of the evidence” basis to approve these products. This has lead the scientists to develop novel and innovative methods to demonstrate similarity of structure with the innovator or what is routinely termed as Reference Listed Drugs or RLDs.

There is a large unmet need in the art of protein engineering and biopharmaceutical manufacturing for methods to assess protein structural similarity in a thermodynamic steady state to assure safety of biomolecules. The instant disclosure fulfills this need by providing a non-destructive method of detecting fluorescence under thermodynamic stress conditions induced by osmotic and dielectric changes.

### SUMMARY

The present disclosure provides methods to assess structural similarity of a first biomolecule and a second biomolecule by detecting one or more responses of the first and second biomolecule to thermodynamic stress conditions induced by osmotic and dielectric changes including, detecting a shift in fluorescence emission and/or a change in the intensity of the emission. In one embodiment of the method, the disclosure produces a gentle stress on the protein structure by altering the osmolality or dielectric conditions in the surrounding medium resulting in a change in the binding of water molecules and perhaps an altered binding of ions with functional groups such as tryptophan, phenylalanine and tyrosine. Two sources of the same protein are then compared by the shift in the spectra and changes in the intensity of emission under various conditions of change in osmolality and dielectric conditions, including the change in ionic strength. A similar change under different stress conditions signifies a high similarity of structure.

The method of the disclosure is applicable to the analysis of any functional protein comprising at least one fluorophor including tryptophan, tyrosine or phenylananine, the aromatic amino acid capable of providing a fluorescent response.

The method exploiting the fluorescent properties of the three aromatic amino acids can be used to assess structural similarity of complex proteins or protein mixtures. In one embodiment of the disclosure, the method can be applied to assessing the biosimilarity of polyclonal antibody preparations, monoclonal antibodies, antibody fragments, such as Fahs; antibody derived constructs, such as scFv and single antibody domains; protein therapeutics, which may be enzymes, industrial enzymes, peptides, and protein digests; and any variant or derivative thereof, provided that these biomolecules contain aromatic amino acid capable of providing a fluorescent response.

In another aspect of the disclosure, the method uses an osmotic stress analysis (OSA) to alter the structure of proteins to demonstrate structural similarity based on the assumption that if the changes under an applied stress are the same, then the initial structure should also be the same. This method of the disclosure may be applied to any aspect of protein product research or development where information on protein structure is a useful parameter. In various aspects of the disclosure, the method is used to determine intrinsic structure during screening of protein variants or alternate candidates produced in early stages of the selection process, determine intrinsic structure of candidates in the final selection process, determine sample structure changes under different formulations in pharmaceutical development, or determine sample structure under different storage and stress conditions.

In yet another aspect of the disclosure, the method uses a dielectric stress caused by changes in the concentration of a surfactant to alter the structure of proteins to demonstrate structural similarity based on the assumption that if the changes under an applied stress are the same then the initial structure should also be the same. This method of the disclosure may be applied to any aspect of protein product research or development where information on protein structural structure is a useful parameter. In various aspects of the disclosure, the method is used to determine intrinsic structure during screening of protein variants or alternate candidates produced in early stages of the selection process, determine intrinsic structure of candidates in the final selection process, determine sample structure changes under different formulations in pharmaceutical development, or determine sample structure under different storage and stress conditions.

In another aspect of the disclosure, the method is used to demonstrate biosimilarity of recombinant therapeutic proteins.

In another aspect of the disclosure, the method is used to establish comparable safety of recombinant therapeutic proteins.

### BRIEF DESCRIPTION OF THE OF THE DRAWINGS

The foregoing summary, as well as the following detailed description of the disclosure, will be better understood when read in conjunction with the appended figures. For the purpose of illustrating the disclosure, shown in the figures are embodiments which are presently preferred. It should be understood, however, that the disclosure is not limited to the precise arrangements, examples and instrumentalities shown.

FIG. 1 shows the effect of change in the osmolality of the solution.

FIG. 2 shows the effect of a 6-fold (0.004% to 0.024% w/v) increase in the concentration of polysorbate 80 on the fluorescence characteristics of filgrastim in TPI-Filgrastim (Theragrastim™) and NEUPOGEN®.

FIG. 3 shows the results for TPI-PEG-Filgrastim with increasing tonicity.

FIG. 4 shows the results for TPI-PEG-Filgrastim with increasing PS-80 concentration.

FIG. 5 shows the results for HSA with increasing tonicity.

FIG. 6 shows the results for HSA with increases in PS-80 concentration.

FIG. 7 shows the results for Lysozyme with increases in tonicity.

FIG. 8 shows the results for Lysozyme with increases in PS-80 concentration.

### DETAILED DESCRIPTION

### Definitions

A “biomolecule” means a chemical entity produced by a biological process that may comprise a protein, either natural or recombinant.

A “protein” means a peptide or polypeptide molecule that may comprise a single subunit or multiple subunits.

The terms “structurally similar” and “structural similarity” with regard to a biomolecule are used interchangeably herein and refer to one or more structural properties of a biomolecule that are similar between a first biomolecule and a second biomolecule (e.g., a reference biomolecule) including, for example, fluorescence emission wavelength and/or intensity of fluorescence of a solution comprising the biomolecule. A first biomolecule may be considered structurally similar to a second biomolecule where one or more structural properties of the first and second biomolecule are 100%, 99%, 98%, 97%, 96%, 95%, 94%, 93%, 92%, 91%, 90%, 85%, 80%, or 75% identical. In some embodiments, a first biomolecule is considered structurally similar to a second biomolecule where a first structural property is 100%, 99%, 98%, 97%, 96%, 95%, 94%, 93%, 92%, 91%, 90%, 85%, 80%, or 75% identical between the first and second biomolecule and a second structural property is 100%, 99%, 98%, 97%, 96%, 95%, 94%, 93%, 92%, 91%, 90%, 85%, 80%, or 75% identical between the first and second biomolecule.

“Osmolyte” means an agent known to alter the osmolality of a solution and thus capable of disrupting covalent interactions within a protein, including hydrogen bonds, electrostatic bonds, Van der Waals forces, hydrophobic interactions, or disulfide bonds and also bonding with water molecules. Examples of osmolytes include polyethylenes and buffers, salts, urea, non-ionic and ionic detergents, acids (e.g. hydrochloric acid (HCl), acetic acid (CH3COOH), halogenated acetic acids); and hydrophobic molecules (e.g., phosopholipids).

“Osmotic Stress Analysis (OSA)” means a change in the composition of the buffer that results in an altered osmolality and the effect of this change in observed in the behavior of the protein.

“Biosimilarity” means a demonstration of similarity in the structure, clinical response, toxicity and side effects in a comparative mode between a newly developed drug and an innovator product or reference listed drug (RLD).

“Recombinant product” means a biomolecule produced in a cell or organism whose DNA has been modified by inserting or combining a gene sequence responsible for expressing the biomolecule.

Assessment of protein structure can be viewed as the ultimate test of the safety of biosimilar molecules. Recombinant proteins expressed in genetically modified organisms may produce structural variations that are beyond the primary or secondary structures and even beyond tertiary structures; how a protein molecules associates with other entities, charged or otherwise in a solution often determines its activity, toxicity and the side effects.

The instant disclosure probes differences in the folded states as affected by an applied osmotic stress resulting from higher concentration of osmolytes, more specifically ionic osmolytes. Increasing the osmolality modifies the boundary of molecules surrounding the biomolecules without affecting the native structure. The choice of osmolyte is also significant since the goal is to bring as few changes to the molecule and for this reason, such commonly used osmolytes as polyethylene glycol and glycerol are avoided. The products tested were in their native buffer solution and only the concentration of ions in the buffers was modulated to achieve a several-fold increase in the osmolality. This is thus the gentlest way to probe proteins and provides a thermodynamically stable assessment of differences in the structure. However, as a general principle, any osmolyte, ionic or otherwise would show a demonstrable effect on the fluorescence if the protein contains fluorophors.

Fluorescence is the result of a three-stage process that occurs in certain molecules called fluorophores. The entire fluorescence process is cyclical. Unless the fluorophore is irreversibly destroyed in the excited state (an important phenomenon known as photobleaching), the same fluorophore can be repeatedly excited and detected. The fact that a single fluorophore can generate many thousands of detectable photons is fundamental to the high sensitivity of fluorescence detection techniques. For polyatomic molecules in solution, the discrete electronic transitions are replaced by rather broad energy spectra called the fluorescence excitation spectrum and fluorescence emission spectrum, respectively. The bandwidths of these spectra are parameters of particular importance for applications in which two or more different fluorophores are simultaneously detected. With few exceptions, the fluorescence excitation spectrum of a single fluorophore species in dilute solution is identical to its absorption spectrum. Under the same conditions, the fluorescence emission spectrum is independent of the excitation wavelength, due to the partial dissipation of excitation energy during the excited-state lifetime. Fluorescence intensity is quantitatively dependent on the same parameters as absorbance—defined by the Beer-Lambert law as the product of the molar extinction coefficient, optical path length and solute concentration—as well as on the fluorescence quantum yield of the fluorophore, the excitation source intensity and fluorescence collection efficiency of the instrument. In dilute solutions or suspensions, fluorescence intensity is linearly proportional to these parameters. Protein folding is the reaction by which a protein adopts its native 3D structure. The native structure is the functional state of the protein. Folding happens in several steps, in a simplistic manner, first is formation of the secondary structure (2D) followed by acquisition of the tertiary structure arrangement (3D), and sometime a further quaternary structure (4D) organization in the case of oligomeric complex proteins. The 2D of a protein can be monitored by Circular Dichroism (CD) whereas the 3D structure can be tracked down using fluorescence spectroscopy, in particular intrinsic protein fluorescence.

There are three amino acids with intrinsic fluorescence properties, phenylalanine (Phe), tyrosine (Tyr) and tryptophan (Trp) but only tyrosine and tryptophan are of greater use experimentally because their quantum yields (emitted photons/exited photons) is high enough to give a good fluorescence signal. The instant disclosure exploits the presence of these three aromatic amino acids found in almost all recombinant proteins. Tryptophan (IUPAC-IUBMB abbreviation: Trp or W; IUPAC abbreviation: L-Trp or D-Trp; sold for medical use as Tryptan) encoded in the standard genetic code as the codon UGG. Tyrosine (abbreviated as Tyr or Y) or 4-hydroxyphenylalanine, encoded as codons UAC and UAU. It is a non-essential amino acid with a polar side group. Phenylalanine (abbreviated as Phe or F) is an a-amino acid, an essential amino acid classified as nonpolar because of the hydrophobic nature of the benzyl side chain. L-Phenylalanine (LPA) is an electrically neutral amino acid, one of the twenty common amino acids used to biochemically form proteins. The codons for L-phenylalanine are UUU and UUC. Phenylalanine is a precursor for tyrosine.

So this technique is based on proteins having either Trp or Tyr or both, which is generally the case for most proteins; therefore, the instant disclosure is not limited to any special class or type of proteins. These amino acids have specific excitation and emission properties (Table 1).

TABLE 1
Fluorescent Characteristics of the Aromatic Amino Acids

| | Absorption | | Fluorescence | |
|---|---|---|---|---|
| | Wavelength | | Wavelength | Quantum |
| Amino Acid | (nm) | Absorbtivity | (nm) | Yield |
| Tryptophan | 280 | 5,600 | 348 | 0.20 |
| Tyrosine | 274 | 1,400 | 303 | 0.14 |
| Phenylalanine | 257 | 200 | 282 | 0.04 |

For an excitation wavelength of 280 run, both Trp and Tyr will be excited. To selectively excite Trp only, 295 run wavelengths must be used. Those residues can be used to follow protein folding because their fluorescence properties (quantum yields) are sensitive to their environment which changes when a protein folds/unfolds. In the native folded state, Trp and Tyr are generally located within the core of the protein, whereas in a partially folded or unfolded state, they become exposed to solvent. In a hydrophobic environment (buried within the core of the protein), Tyr and Trp have a high quantum yield and therefore high fluorescence intensity. In contrast in a hydrophilic environment, (exposed to solvent) their quantum yield decreases leading to low fluorescence intensity. For Trp residue, there is strong stoke shifts dependent on the solvent, meaning that the maximum emission wavelength of Trp will differ depending on the Trp environment. There are several means to unfold a protein based on the disturbance of the weak interactions that maintains the protein folded (hydrogen bonding, electrostatic interactions, hydrophobic interactions).

The most common ways of unfolding a protein are chaotropic agents (urea, guanidium hydrochloride), the change of pH (acid, base) or the rise of temperature. It is possible to study either steady state or kinetic state of protein unfolding. For example, the protein is unfolded by increasing temperature, so at each temperature the protein undergoes unfolding and reaches an equilibrium state corresponding to a partially folded or fully unfolded state depending on the conditions. Fluorescence intensity (FI) will change upon unfolding as well as the maximum emission wavelength (λmax) if Trp is used as a monitor. Following the change of this parameter (FI or λmax) the unfolding curve is generated by plotting FI=f(temperature) or λmax=f(temperature). Those kinds of studies are steady state studies. For kinetic studies, the protein is put at one temperature and its unfolding reaction is followed in time. Here again the change in either FI or λmax is measured but in time.

Water plays a central role in a wide range of biomolecular processes, from protein folding, stability, and denaturation to physiological regulation and allosteric effects. Water is involved in these processes in a variety of ways, ranging from direct bridging to collective effects (such as hydrophobic effects). The enumeration of water molecules is crucial in order to understand how biomolecular processes work. Osmotic stress analysis (OSA) aims to estimate the number of water molecules adsorbed (or released) as a result of biomolecular processes. To do so, osmolytes (such as glycerol and polyethylene glycol, known also as protein stabilizers) are added to the system. Because protein-stabilizing osmolytes, preferentially excluded from protein surfaces, are not accessible to cavities, grooves, channels, or pockets formed by biomolecules, these regions are subject to osmotic stress. Osmotic stress and the accompanied change of water activity modulate the equilibrium of the process, and the number of waters adsorbed upon the reaction in the absence of osmolytes is enumerated by measuring the change of equilibrium constant with respect to osmotic pressure. The underlying assumption is that osmolytes are “inert”: they neither interact nor act directly on macromolecules because they are excluded. OSA was first applied to hemoglobin: about 65 water molecules are assumed adsorbed upon the transition from the T state to the R state. This estimation is consistent with the change in buried surface area. Since then, OSA has been applied to various biomolecular processes, including ion channels, DNA-protein, and carbohydrate-protein interactions.

It has been demonstrated in the present disclosure that using osmotic stress analysis (OSA) as a tool, that the biosimilarity of protein samples from different samples can be established. Water molecules involved in therapeutic proteins play several significant roles. For example, the interactions governing protein folding, stability, recognition, and activity are mediated by hydration. Using small-angle neutron scattering coupled with osmotic stress studies have investigated the hydration of lysozyme and guanylate kinase (GK), in the presence of solutes. By taking advantage of the neutron contrast variation that occurs upon addition of these solutes, the number of protein-associated (solute-excluded) water molecules can be estimated from changes in both the zero-angle scattering intensity and the radius of gyration. Polyethylene glycol is used to produce osmotic stress and effect of stress produced varies with its molecular weight. This sensitivity has been exploited to probe structural features such as the large internal GK cavity. For GK, small-angle neutron scattering was complemented by isothermal titration calorimetry with osmotic stress to also measure hydration changes accompanying ligand binding.

The influence of solvation on the rate of quaternary structural change has been reported using human hemoglobin, an allosteric protein in which reduced water activity destabilizes the R state relative to T. Nanosecond absorption spectroscopy of the heme Soret band was used to monitor protein relaxation after photo dissociation of aqueous HbCO complex under osmotic stress induced by the nonbinding cosolute polyethylene glycol (PEG). Photolysis data analyzed globally for six exponential time constants and amplitudes as a function of osmotic stress and viscosity are used to show increases in time constants associated with geminate rebinding, tertiary relaxation, and quaternary relaxation were observed in the presence of PEG, along with a decrease in the fraction of hemes rebinding carbon monoxide (CO) with the slow rate constant characteristic of the T state. An analysis of these results along with those obtained by others for small cosolutes showed that both osmotic stress and solvent viscosity are important determinants of the microscopic R [symbol] T rate constant. The size and direction of the osmotic stress effect suggests that at least nine additional water molecules are required to solvate the allosteric transition state relative to the R-state hydration, implying that the transition state has a greater solvent-exposed area than either end state.

The thermal stability of nucleic acid structures is perturbed under the conditions that mimic the intracellular environment, typically rich in inert components and under osmotic stress. Studies describe the thermodynamic stability of DNA oligonucleotide structures in the presence of high background concentrations of neutral cosolutes. Small cosolutes destabilize the base pair structures, and the DNA structures consisting of the same nearest-neighbor composition show similar thermodynamic parameters in the presence of various types of cosolutes. The osmotic stress experiments reveal that water binding to flexible loops, unstable mismatches, and an abasic site upon DNA folding are almost negligible, whereas the binding to stable mismatch pairs is significant. These studies using the base pair-mimic nucleosides and the peptide nucleic acid suggest that the sugar-phosphate backbone and the integrity of the base pair conformation make important contributions to the binding of water molecules to the DNA bases and helical grooves. The study of the DNA hydration provides the basis for understanding and predicting nucleic acid structures in non-aqueous solvent systems.

Membrane deformation and tension potentially affect the conformational energetics of membrane proteins such as rhodopsin though non-specific lipid-protein interactions. The question how membrane deformation can alter these protein-lipid interactions and thus affect membrane protein function has been studied through usage of osmolytes and dehydration to observe deformation in DMPC-d54 membranes via solid-state 2H NMR. Measured order parameters allow deformations to be accessed at the molecular level. Stresses from dehydration and osmotic pressure are thermodynamically equivalent because the change in chemical potential when transferring water from the inter-lamellar space to the bulk water phase corresponds to an induced pressure. Due to equivalence of the two stresses, there is a direct relationship to membrane hydration to an applied osmotic pressure via the order parameters. These findings demonstrate the ability to change membrane structure in a controlled manner for the investigation of pressure and hydration sensitivity of membrane proteins.

In essence, given the significant role played by water and connecting with the activity of water in various thermodynamic states, the validity of osmotic stress strategy can be revisited to study macromolecular biomolecules. Water can fill the obligatory space, it dominates nearest non-specific interactions between large surfaces, as it can be a reactant modulating conformational change; all this in addition to its more commonly perceived static role as an integral part of stereospecific intra-molecular structure.

Osmotic stress is used to measure solvation changes that accompany the conformational changes of an active enzyme. For hexokinase, both the equilibrium dissociation constant and the kinetic Michaelis-Menten constant for glucose vary linearly, and to the same extent, with the activity of water in the protein medium, as adjusted with large molecular weight (>2000) osmolytes. The variation over the whole osmotic pressure range studied indicates that glucose binding is accompanied by the release of at least 65±10-water molecules, and this is reversed on enzyme turnover. The results indicate that near the physiological range of pressures the number may be higher. Most of this water, which behaves like an inhibitor, likely comes from the cleft, which is induced to close around the substrate. Such large dehydration/rehydration reactions during turnover imply a significant contribution of solvation to the energetics of the conformational changes. Osmotic stress is a method of general applicability to probe water's contribution to functioning molecules.

Protein folding and conformational changes are influenced by protein-water interactions and, as such, the energetics of protein function are necessarily linked to water activity. Studies on the helix-coil transition using polyglutamic acid as a model system are reported to investigate the importance of hydration to protein structure by using the osmotic stress method combined with circular dichroism spectroscopy. Osmotic stress is applied using polyethylene glycol, molecular weight of 400, as the osmolyte. The energetics of the helix-coil transition under applied osmotic stress allows calculation of the change in the number of preferentially included water molecules per residue accompanying the thermally induced conformational change. It is reported that osmotic stress raises the helix-coil transition temperature by favoring the more compact alpha-helical state over the more hydrated coil state. The contribution of other forces to alpha-helix stability also are explored by varying pH and studying a random copolymer, poly(glutamic acid-r-alanine). Evidence is available of the influence of osmotic pressure on the peptide folding equilibrium and studies on protein folding in vitro demonstrate that the osmotic pressure, in addition to pH and salt concentration, should be controlled to better approximate the crowded environment inside cells.

The addition of polyethylene glycol (PEG), of various molecular weights, to solutions bathing yeast hexokinase increases the affinity of the enzyme for its substrate glucose. The results can be interpreted on the basis that PEG acts directly on the protein or indirectly through water activity. The nature of the effects suggests that PEG's action is indirect. Interpretation of the results as an osmotic effect yields a decrease in the number of water molecules, Δ Nw, associated with the glucose binding reaction. The Δ Nw is the difference in the number of PEG-inaccessible water molecules between the glucose-bound and glucose-free conformations of hexokinase. At low PEG concentrations, Δ Nw increases from 50 to 326 with increasing MW of the PEG from 300 to 1000, and then remains constant for MW-PEG up to 10,000. This suggests that up to MW 1000, solutes of increasing size are excluded from ever-larger aqueous compartments around the protein. Three hundred and twenty-six waters are larger than is estimated from modeling solvent volumes around the crystal structures of the two hexokinase conformations. For PEGs of MW>1000, Δ Nw falls from 326 to about 25 waters with increasing PEG concentration, i.e., PEG alone appears to “dehydrate” the unbound conformation of hexokinase in solution. Remarkably, the osmotic work of this dehydration would be on the order of only one k T per hexokinase molecule. Under thermal fluctuations, hexokinase in solution has a conformational flexibility that explores a wide range of hydration states not seen in the crystal structure.

The structures at protein-water interface, i.e., the hydration structure of proteins, have been investigated by cryogenic X-ray crystal structure analyses. Hydration structures appeared far clearer at cryogenic temperature than at ambient temperature, presumably because cooling quenched the motions of hydration water molecules. Based on the structural models obtained, the hydration structures were systematically analyzed with respect to the amount of water molecules, the interaction modes between water molecules and proteins, the local and the global distribution of them on the surface of proteins. The standard tetrahedral interaction geometry of water in bulk retained at the interface and enabled the three-dimensional chain connection of hydrogen bonds between hydration water molecules and polar protein atoms. Large-scale networks of hydrogen bonds covering the entire surface of proteins are highly flexible to accommodate to the large-scale conformational changes of proteins and seemed to have great influences on the dynamics and function of proteins.

Water in close proximity to the protein surface is fundamental to protein folding, stability, recognition and activity. Protein structures studied by diffraction methods show ordered water molecules around some charged, polar, and non-polar (hydrophobic) amino acids, although the later are only observed when they are at the interface between symmetry related molecules in the crystal. Water networks surrounding the protein have been observed for small proteins. Crystallographically observed water molecules are referred to as bound structural water molecules. During crystallographic data analysis, bound water molecules are often treated as though they belong to the protein. Recent developments in the treatment of the bulk solvent contribution to the low order diffraction data allow a better evaluation of the surface structure of the protein and a better localization of bound waters. The mobility of bound waters is studied by means of temperature and occupancy factors. The bulk solvent has relatively large disorder (liquid like), which is represented by liquidity factors. Within this context water layers surrounding the protein have little mobility.

Conformational instability refers not only to unfolding, aggregation, or denaturation but also to subtle changes in localized protein domains and the alteration of enzyme catalytic properties that may result from buffer-component binding, proton transfer, and metal or substrate binding effects directly or indirectly mediated by buffers or by buffers themselves acting as pseudo-substrates. Salts can affect protein conformation to the extent that the anions or cations of the salt could be potential buffer components. When the salt concentration is much larger than that of the buffer, the salt becomes the effective buffer in the reaction. The mechanisms or combinations thereof by which buffers may cause protein stabilization (or destabilization) are complex and not well understood. The problem is compounded by the inability to definitively differentiate between various protein stabilization mechanisms and the small free energies of stabilization of globular proteins. There is no prior art that definitively address some of these issues as they relate to buffers used in the formulation of proteins. The effect of buffers that may be used in the extraction, purification, dialysis, refolding, or assay of proteins on protein conformation is not known. Observations are however made such as the aggregation of lyophilized natriuretic peptide (ANP, pl 10) was significantly reduced when the concentration of acetic acid buffer at pH 4.0 was increased from 5 to 15 mM before lyophilization. The mechanism of aggregation has been attributed to alkali-induced elimination from the disulfide linkage to form a free thiolate ion. The thiolate anion subsequently undergoes thiol-disulfide interchange with ANP to form the disulfide-linked multimers. However, it is not apparent why a phase transition of ostensibly incompletely crystallized mannitol after lyophilization from a glass to a crystal upon storage would trigger an increase of local pH in the lyophilized product (that was attributed to the generation of thiolate ions).

Protection against aggregation caused by mechanical stress is widely suggested. For example, the stability of G-CSF (granulocyte colony stimulating factor) toward agglomeration has been measured by light scattering at 360 nm over a range of pH values in three different buffer solutions (80 mM). The stabilization of G-CSF against denaturation induced by mechanical stress differs depending on buffer type and pH. Buffers can alter protein-surfactant binding characteristics and thereby change protein conformation. Results of a study showed that increasing the concentration of sodium phosphate buffer (pH around 7.1) from 10 to 100 mM increased the amount of sodium dodecyl sulfate (SDS) bound to reduced-carboxyamidomethylated bovine serum albumin (RCAM-BSA) from 1.0 to 2.2 gig. In another study, a coadsorbed multilayer of SDS and lysozyme formed in the transitional binding regime at pH 6.9 in 8.8 mM phosphate buffer but not at pH 5.0 in 5.0 mM acetate buffer. The binding isotherms showed that approximately the same number of molecules of SDS bound to lysozyme between the onset and completion of transitional binding at both pH values. The greater aggregation tendency in the phosphate buffer is likely caused by a more effective charge screening by the divalent phosphate ion than by the univalent acetate ions.

Historically, buffers are not generally believed to have profound effects on the tertiary and quaternary structures of proteins. It is important to realize that buffers perturb protein conformational stability because of a complex interplay between various effects rather than by stand-alone mechanisms. For example, some of the antioxidant effects of Good's buffers may arise because of their metal binding ability. Binding or substrate effects may predominate the interaction of buffers with proteins at low buffer concentrations; electrostatic charge screening may dominate at intermediate concentrations and kosmotropic/chaotropic effects may prevail at higher concentrations. The contribution of charge repulsion by buffer anions to thiol-disulfide exchange reactions may vary with the degree of buffer deprotonation, as can the contribution of buffer to amide exchange rates.

Because of the extremely diverse structure and related properties of proteins, it may not be possible to predict a priori the “best” buffer for any given protein molecule. However, some correlative generalizations can be attempted-recognizing that these may not necessarily be causative in nature. Buffers that may best protect a given protein from a variety of denaturing stresses should possess the following attributes: ability to incorporate the electron-donating and electron accepting sites on one molecule (i.e., be zwitterionic); preferentially be excluded from the protein domain (i.e., increase the surface tension of water) and incorporate kosmotropic ions, such as sulfate, phosphate, magnesium, lithium, zinc, and aluminum; possess a low heat of ionization; decrease the mobility of water molecules; cause negligible change in the denaturation Gibbs energy for that protein; not undergo or catalyze complexation with the carbohydrate part of the glycosylated protein; inhibit the nucleophilic attack of the thiolate anion on disulfide links, thus preventing thiol-disulfide interchange; unless intended, not act as a substrate for the enzyme, not catalyze metal mediated redox reactions or alter surfactant binding characteristics to the protein; not render the protein more susceptible to mechanical stress; not cause an increase in the proton amide exchange rate for the protein residues with the buffer vis-a-vis an “inert” buffer medium.

The Dielectric Constant, or permittivity, e, is a dimensionless constant that indicates how easy a material can be polarized by imposition of an electric field on an insulating material. The constant is the ratio between the actual material ability to carry an alternating current to the ability of a vacuum to carry the current. The dielectric constant can be expressed as:

∈=∈ₛ/∈₀, where,

∈=the dielectric constant;

∈ₛ=the static permittivity of the material; and

∈₀=vacuum permittivity.

The dielectric constant of water is about 80, of vacuum and mercury around 1. It is highly dependent on temperature.

Surfactants like polysorbate 20 and 80, also known as Tween® 20 or 80, are commonly used excipients in formulations of therapeutic proteins. The main function of the amphiphilic polysorbates is to prevent protein adsorption at liquid-liquid, liquid-solid or liquid-air interfaces, which can lead to surface-induced denaturation and aggregation. A protective effect of polysorbates on protein stability has been shown during freeze-thawing, freeze-drying, mechanical stress (e.g. agitation, shaking or stirring and reconstitution of dried protein preparations as well as for formulations containing silicone oil droplets. However, polysorbates can also negatively affect stability, e.g., at quiescent conditions during long-term stability. Furthermore, polysorbates can undergo various degradation reactions, which can lead to a loss of its stabilizing properties and chemical modifications of proteins, such as oxidation.

Non-ionic surfactants protect proteins from surface (e.g., agitation or shaking) and stress induced aggregation (e.g., freezing, lyophilization, and reconstitution). Surfactants act by competing with proteins for contain surface, air/water interface, ice/water interface, or any other solid surfaces and prevent non-specific adsorption and adsorption induced denaturation and subsequent aggregation. In some cases, surfactants also prevent aggregation by serving as chaperones and foster protein folding and refolding (e.g., induction of folding of membrane proteins by surfactants). However, the commonly used polysorbates may degrade by oxidation or hydrolysis, and their degradation products may exert varying effects on protein stability. Additionally, it can be difficult to control the level of surfactants in the formulation due to complex behaviors during membrane filtration steps.

Almost 70% of the marketed monoclonal antibody formulations contain polysorbate 20 or polysorbate 80 as stabilizing excipients. Within those commercial preparations, the polysorbate concentrations range between 0.001% (w/v) polysorbate 80 (Reopro®) and 0.16% (w/v) polysorbate 20 (Raptiva®), with most formulations containing about 0.005 to 0.02% polysorbate 20 or 80. One difference between the polysorbates is the lower critical micelle concentration of polysorbate 80 (ca. 0.0017% (w/v)) compared to polysorbate 20 (ca. 0.007% (w/v). This property can therefore be used to create a dielectric stress in the solutions of therapeutic proteins.

Often the surroundings of a thermodynamic system may also be regarded as another thermodynamic system. In this view, one may consider the system and its surroundings as two systems in mutual contact, with long-range forces also linking them. The enclosure of the system is the surface of contiguity or boundary between the two systems. In the thermodynamic formalism, that surface is regarded as having specific properties of permeability. For example, the surface of contiguity may be supposed to be permeable to electrical charges, allowing an extension of the dielectric property of the surrounding thermodynamic system. As an example, G-CSF (Granulocyte Colony Stimulating Factor) was used in this disclosure to demonstrate the utility of the invented method. Recombinant human G-CSF has 175 residues and it is expressed in E. coli. The protein has an amino acid sequence that is identical to the natural sequence predicted from human DNA sequence analysis, except for the addition of an N-terminal methionine necessary for expression in E coli.

G-CSF has three tyrosines, six phenylalanines and two tryptophans, the aromatic amino acids capable of fluorescing. Since both phenylalanine and tryptophan are nonpolar, their interaction with water molecules or with species of a buffer solution occurs by a different mechanism than the interaction of tyrosine, which is polar. Water and other entities found in the formulation of the products of G-CSF tested may bind or interact with both polar and non-polar amino acids. When we consider how the structuring of water make this highly polar entity a non-polar entity, we realize that each of the three aromatic amino acids are important in establishing a robust protocol for protein structure validation.

The method of the present disclosure can thus be used advantageously to provide information about the chemical structure of proteins or the method can be used empirically to rank and select among a series of variants or varied preparations on the basis of their overall structural compliance with a reference protein as may be required in the process development of the manufacturing of recombinant proteins and monoclonal antibodies where minor changes in the in process controls may affect their structure.

In one embodiment, acetate buffer was used as source of ionic strength, but this is not limited to any specific buffer species since the osmotic stress can be achieved from various osmolytes including non-ionic osmolytes, such as polyethylene glycols. In other aspects of the disclosure, other osmolytes can thus be substituted for acetate ionic species. Natural osmolytes include trimethylamine N-oxide (TMAO), dimethylsulfoniopropionate, trimethylglycine, sarcosine, betaine, glycerophosphorylcholine, myo-inositol, and taurine. Osmolytes may also be glycerol, polyethylene glycols, buffers, e.g., acetate buffer, salts, urea, non-ionic, ionic detergents, acids and hydrophobic molecules.

In one embodiment, polysorbate 80 was used as a source of modulation of dielectric properties but this is not limited to any specific surfactant since the changes in the dielectric properties can be achieved from various polar and nonpolar components, including surfactants.

The method of determining protein conformation structure and integrity are highly relevant to demonstrating biosimilarity of follow-on proteins and antibodies. Whereas much progress has been made in using standard methods that disclose typical two and three dimensional differences, the problems associated with immunogenicity of proteins requires further study of the fourth dimensional structure of proteins. The association of the functional groups in proteins molecules with components of the media is reported to be a fast method for evaluating structural differences between samples derived from different sources. Osmotic stress produced ideally by increasing the ionic strength of the final formulation buffer provides an ideal solution to an observation that is highly clinically relevant. Other methods of physically or chemically breaking down proteins do not provide the sensitive information needed to fully establish safety of biosimilar products.

Disclosed herein is particularly useful in industrial settings where quantities of active proteins are produced. The method of the present disclosure may also be used as an additional method to discriminate between proteins with other similar properties. By discriminating between proteins on the basis of their thermodynamically stable structure, an alternate parameter for measuring protein structure similarity is achieved. The difference in structures can be measured using either manual or automated methods described above and recording signal strength over time.

Whereas the commercial products tested in the instant disclosure are isotonic when intended for intravenous injection, the instant disclosure uses at least two ranges, one closer to where the product will not cause hemolysis and the other where it will not cause crenation. Beyond these ranges, the product will be unsuitable for administration to humans. To avoid crenation or hemolysis, injections and infusions should have an osmolality as close to plasma as possible. A solution that has the same osmotic pressure as another is called isotonic. In physiology isotonic generally assumes that a solution will have the same osmolality as blood. Large volume infusions should have an osmolality as close to 287-290 mOsm/kg and all injections should have an osmolality as close to the normal range as possible (285-295 mOsm/kg).

Higher osmolality results in loss of water molecules, exposure of the aromatic fluorescent groups and increased fluorescence as expected. The two samples tested showed an identical profile of the shift of fluorescence when the solutions were excited at 284 nm. This provides ample proof of the thermodynamic structural similarity between the two solutions tested.

### EXAMPLES

### Example 1

To test the effect of dielectric and osmotic stress, Theragrastim and Neupogen were subjected to increasing concentrations of acetate and of polysorbate 80 (a nonionic surfactant). The fluorescent properties of the solutions were compared under the same solution conditions. This treatment was conducted after performing a 3-fold dilution of each drug product with 2 M acetate and filgrastim formulation buffer. Thus, the filgrastim concentrations of the test articles were 0.1 mg/mL for vial product and 0.2 mg/mL for syringe product. The concentration of polysorbate 80 ranged from 0.004% to 0.024% (a six-fold change).

TPI filgrastim drug substance used in Theragrastim was diluted from 1.2 mg/mL to 0.6 mg/mL with filgrastim formulation buffer (10 mM acetate, 5% sorbitol, 0.004% polysorbate 80 at pH 4.0). The 0.6 mg/mL filgrastim solution was subsequently diluted three-fold to 0.2 mg/mL with either 200 mM acetate, 5% sorbitol, 0.01% polysorbate 80 at pH 4.0, or 10 mM acetate, 0.004% polysorbate 80 at pH 4.0. The final solution conditions for these test articles were 137 mM acetate, 5% sorbitol, 0.007% polysorbate 80 at pH 4.0, or 10 mM acetate, 1.7% sorbitol, 0.004% polysorbate 80 at pH 4.0, respectively.

Neupogen drug product at 0.6 mg/mL was diluted three-fold to 0.2 mg/mL with either 200 mM acetate, 5% sorbitol, 0.01% polysorbate 80 at pH 4.0, or 10 mM acetate, 0.004% polysorbate 80 at pH 4.0. The final solution conditions for these test articles were 137 mM acetate, 5% sorbitol, 0.007% polysorbate 80 at pH 4.0, or 10 mM acetate, 1.7% sorbitol, 0.004% polysorbate 80 at pH 4.0, respectively.

Appropriate blank solutions were generated prior to acquiring test article spectra by fluorescence spectroscopy and the osmolality of each solution was determined. The osmolality of the 137 mM acetate, 5% sorbitol, 0.007% polysorbate 80 at pH 4.0 solution was determined to be 414 mOsm/kg and the osmolality of the 10 mM acetate, 1.7% sorbitol, 0.004% polysorbate 80 at pH 4.0 was found to be 151 mOsm/kg.

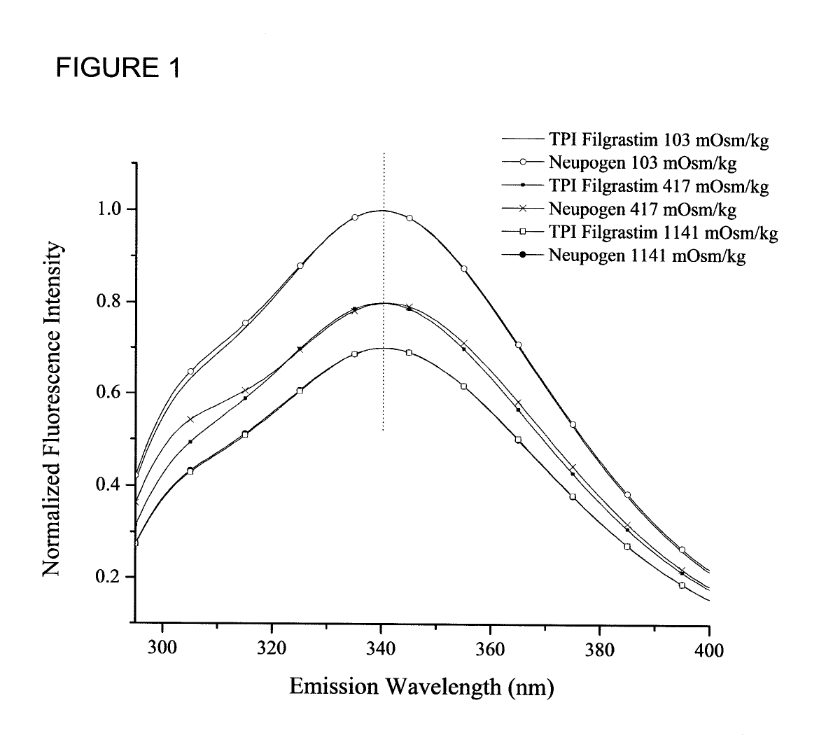

Three fluorescence spectra were acquired on each blank solution using an excitation wavelength of 257 nm while monitoring the emission from 295-400 nm at a scan rate of 100 nm/min at ambient temperature. The average of the three spectra was saved in the instrument's software for automatic subtraction from subsequently acquired sample spectra. FIG. 1 shows the effect of change in the osmolality of the solution. A decrease of approximately 30% in the emission intensity was observed for both TPI-Filgrastim drug substance and NEUPOGEN® (a product of Amgen) as the acetate content was increased to approximately 0.67 M (osmolality of 1141 mOsm/kg), but no significant shifts in the emission wavelengths were observed.

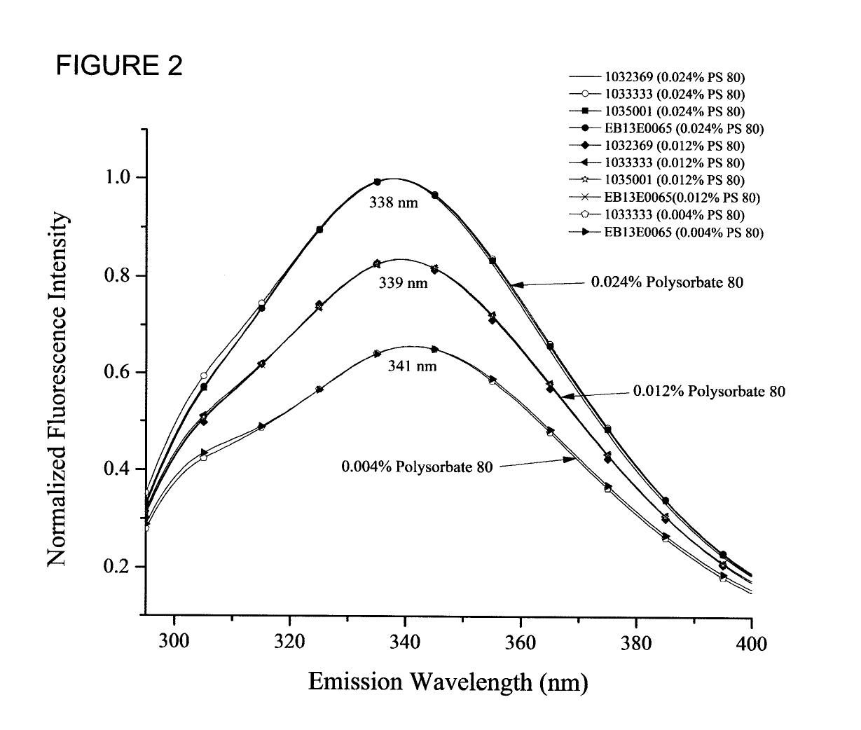

Three fluorescence spectra were acquired on each 0.2 mg/mL Theragrastim and Neupogen sample at ambient temperature using the same parameters used to acquire the blank spectra. The three spectra were automatically averaged in the instrument's software and the blank solution was automatically subtracted from the sample spectra. FIG. 2 shows the effect of a 6-fold (0.004% to 0.024% w/v) increase in the concentration of polysorbate 80 on the fluorescence characteristics of TPI-Filgrastim (Theragrastim™) and NEUPOGEN® (a product of Amgen). A blue shift in the emission wavelength from approximately 341 run to approximately 338 run was observed as the concentration of polysorbate 80 increased. This shift was also accompanied by a significant increase in the fluorescence intensity.

The osmolality of Theragrastim and Neupogen tested ranged from 1141 mOsm/kg to 103 mOsm/kg. Normal human plasma has an osmolality in the range of 285-295 mOsm/kg. Agents that have an osmolality higher than 600 mOsm/kg causes crenation (shriveling up) of red blood cells resulting in significant pain. Solutions that have an osmolality less than about 150 mOsm/kg cause hemolysis (rupture of the red blood cells) and pain at the site of injection.

### Example 2

Three proteins were subjected to osmotic stress and increases in PS-80 concentration (dielectric): (1) TPI-PEG-Filgrastim; (2) Human Serum Albumin (HSA); and (3) Lysozyme. Each protein was prepared in exactly the same manner as described in Example 1 for TPI-Filgrastim. G-CSF contains six Phe (3.4%), two Trp (1.1%) and three Tyr (1.7). HSA contains thirty-one Phe (5.3%), one Trp (0.1%), and eighteen Tyr (3.1%). Lysozyme contains three Phe (2.3%), six Trp (4.7%) and three Tyr (2.3%).

Each protein was analyzed at 0.2 mg/mL. The impact of tonicity was evaluated under each of the following conditions: (1) 10 mM acetate, 1.7% sorbitol, 0.004% PS-80 (104 mOsm/kg); (2) 0.17 M acetate, 5% sorbitol, 0.004% PS-80 (414 mOsm/kg); and (3) 0.67 M acetate, 5% sorbitol, 0.004% PS-80 (1142 mOsm/kg).

The impact of dielectric was evaluated using the high tonicity sample for each protein. The PS-80 concentration was increased to 0.012 and 0.024% (and 0.036% for HSA). Fluorescence emission was measured from 295-400 run using an excitation wavelength of 278 run.

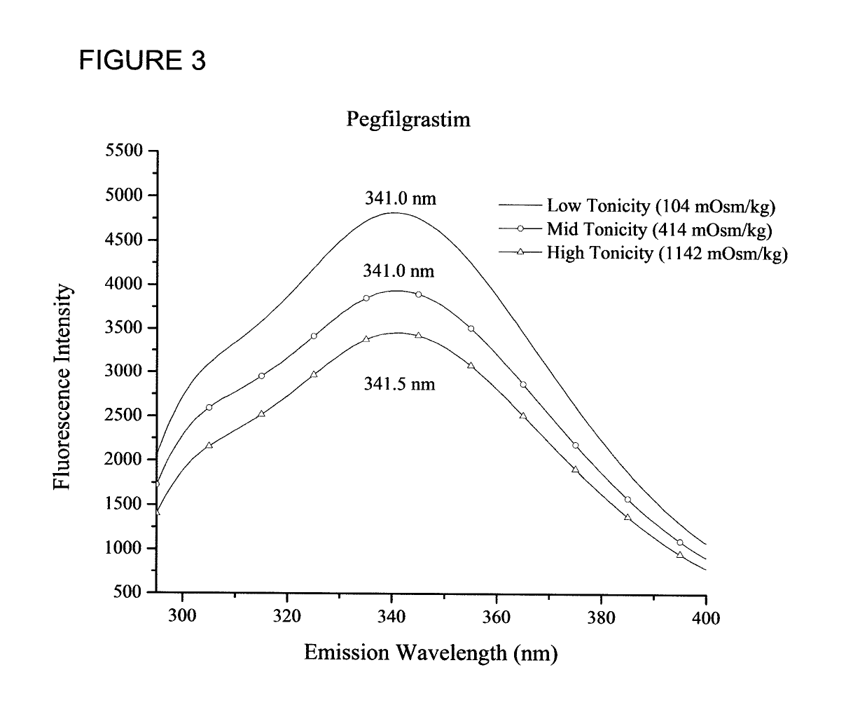

FIG. 3 illustrates the increasing tonicity for TPI-PEG-Filgrastim. Results are similar to those obtained for TPI-Filgrastim. Decreases in emission intensity were observed, but no significant shift in emission maximum was observed.

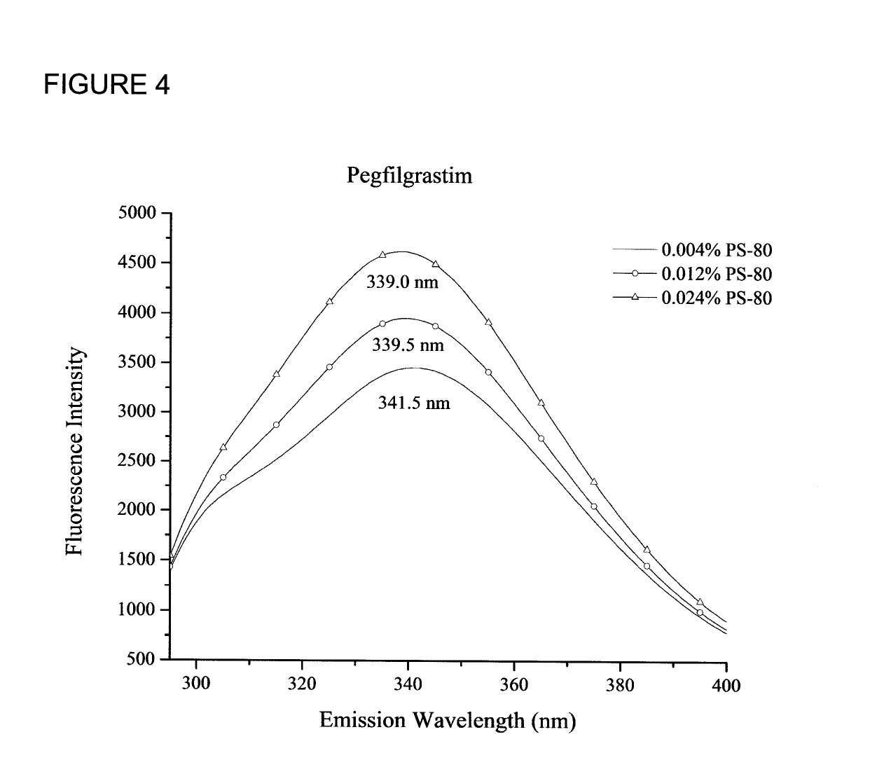

FIG. 4 shows the results for TPI-PEG-Filgrastim under high tonicity conditions with increasing PS-80 concentrations. Results are similar to those obtained for TPI-Filgrastim. Increases in emission intensity were observed concomitant with blue shifts as PS-80 concentration was increased.

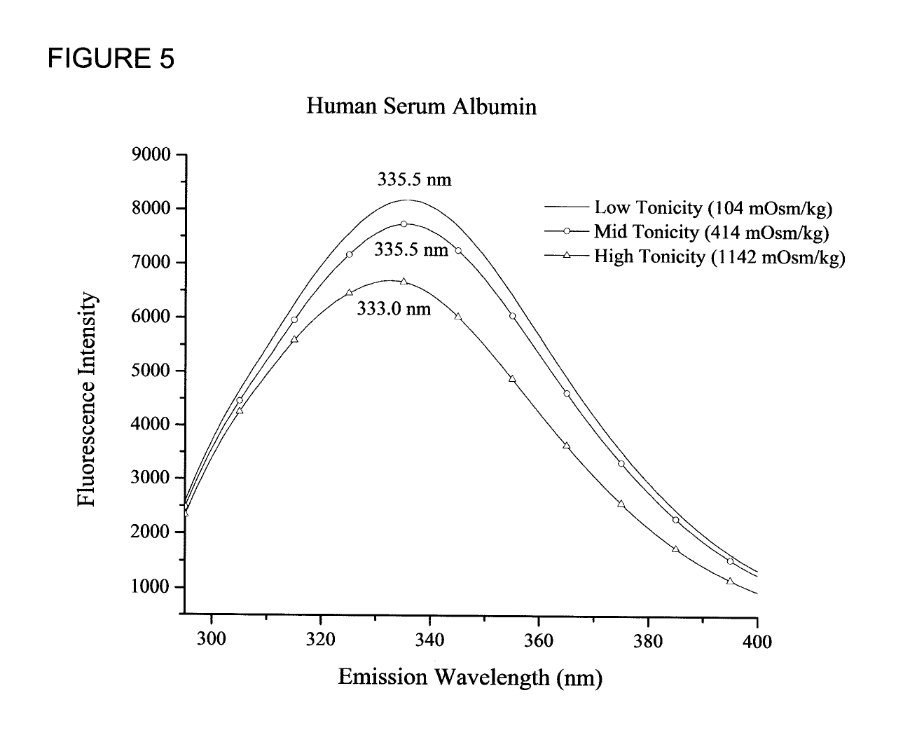

FIG. 5 shows the results for HSA with increasing tonicity. Decreases in emission intensity were observed with increases in tonicity. A significant blue shift was observed as tonicity was increased from 414 to 1142 mOsm/kg.

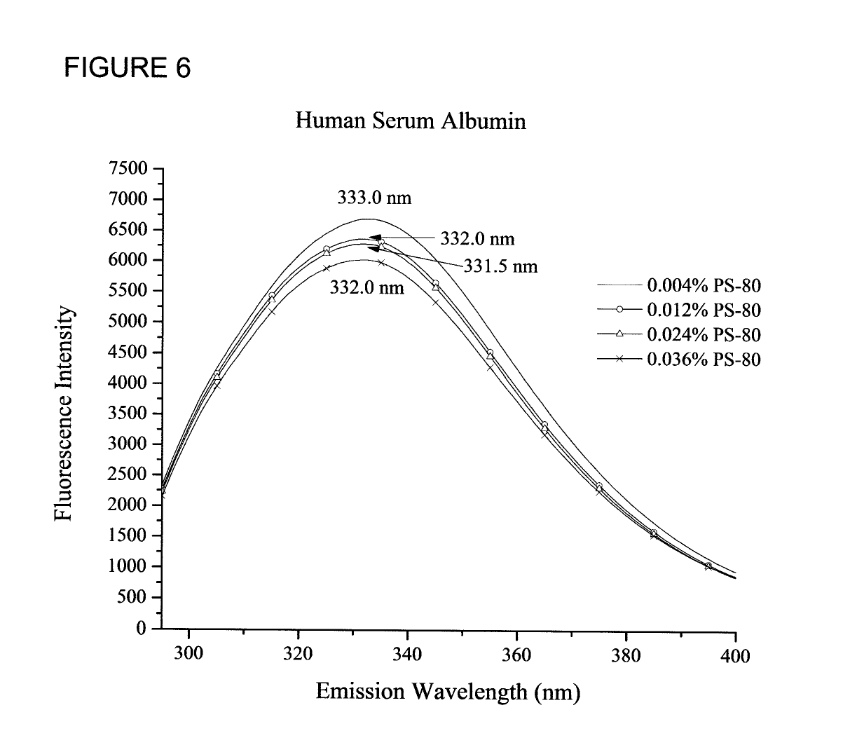

FIG. 6 shows the results for HSA under high tonicity conditions with increases in PS-80 concentration. Decreases in emission intensity were observed with increases in PS-80 concentration. A blue shift of approximately 1 nm was observed with increases in PS-80 concentration.

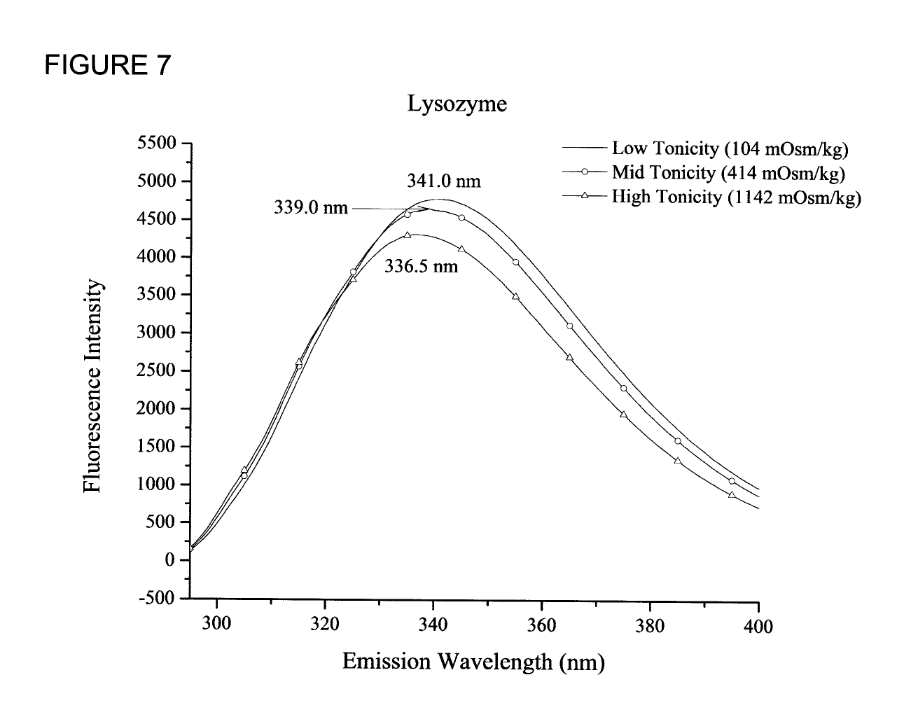

FIG. 7 shows the results for Lysozyme with increases in tonicity. Similar to HSA, decreases in emission intensity were observed with increases in tonicity. A blue shift was also observed with increases in tonicity.

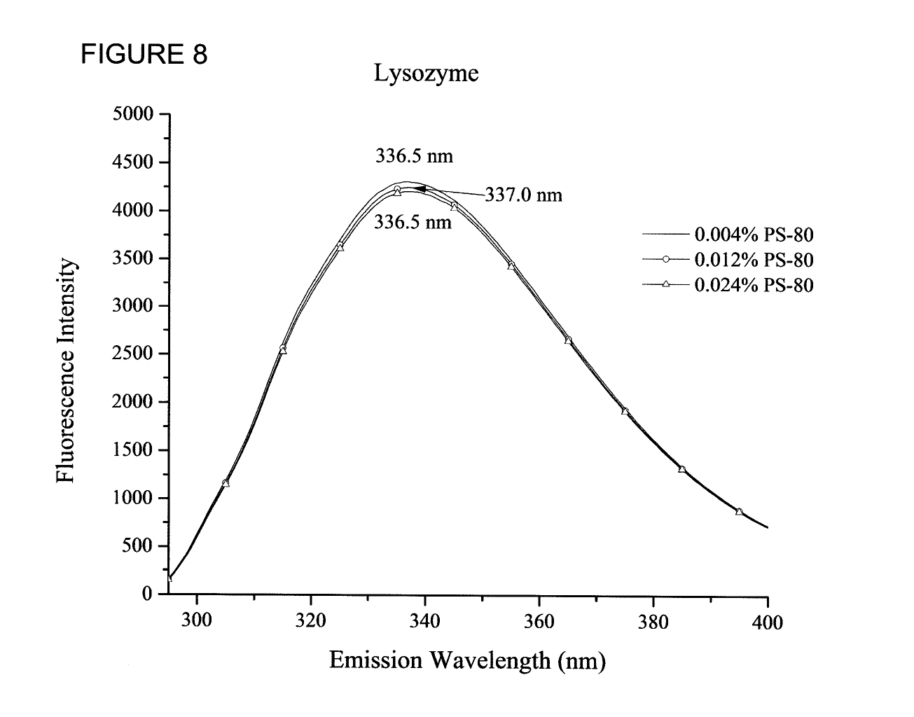

FIG. 8 shows the results for Lysozyme under high tonicity conditions with increases in PS-80 concentration. A slight decrease in emission intensity was observed with increases in PS-80 concentration. No significant shift in emission maxima were observed with increases in PS-80 concentration.

TPI-PEG-Filgrastim showed similar behavior relative to TPI-Filgrastim upon changes in tonicity and dielectric. HSA and lysozyme both manifested decreases in emission intensity with increasing tonicity. No significant shift in the emission maximum was observed for HSA whereas the emission maximum for lysozyme showed a significant blue shift with increases in tonicity. The decreases in fluorescence intensity are attributed to quenching processes, which cause decreases in the fluorescence intensity of a sample. A variety of molecular interactions can result in quenching, including excited-state reactions, molecular rearrangements, energy transfer, ground-state complex formation and collisional quenching. The cause(s) of quenching not only depend on the solution conditions of the sample, but are also dependent upon the conformation of the protein and the accessibility of the aromatic amino acids which provide fluorescence emission. Since lysozyme and HSA have different primary, secondary, and tertiary structures, the accessibility of the aromatics are different relative to filgrastim. Thus, the structural and fluorescence properties change differently for each protein although they are each subjected to the same osmotic and dielectric stresses.

HSA showed a decrease in emission intensity with increases in PS-80 concentration along with ˜1 nm blue shift. Lysozyme showed a modest decrease in emission intensity, but no significant shift in emission maxima with increases in PS-80 concentration. These results are in stark contrast to those obtained for TPI-PEG-Filgrastim and TPI-Filgrastim, which both showed significant increases in fluorescence intensity emission, as well as blue shifts with increases in PS-80 concentration. The degree of changes in fluorescence emission wavelength and intensity as a result of dielectric modifications are dependent upon the accessibility of the fluorescent aromatic amino acids. HSA, for example, contains a unique tryptophan residue that is deeply buried in a hydrophobic binding pocket of the protein (Kragh-Hansen, U., “Molecular aspects of ligand binding to serum albumin”, Pharmacol. Rev. 1981, 33, 17-53; Peters, T., “Serum albumin”, Adv. Protein Chem. 1985, 37, 161-245), whereas lysozyme and filgrastim contain more than one Trp residue with different conformational arrangements and therefore different degrees of solvent accessibility. Thus, the effect of dielectric changes are different for each protein since their structures are not the same.

While the present disclosure has been described and illustrated herein by references to various specific materials, procedures and examples, it is understood that the disclosure is not restricted to the particular combinations of materials and procedures selected for that purpose. Numerous variations of such details can be implied as will be appreciated by those skilled in the art. It is intended that the specification and examples be considered as exemplary, only, with the true scope and spirit of the disclosure being indicated by the following claims. All references, patents, and patent applications referred to in this application are herein incorporated by reference in their entirety.

1-19. (canceled)

20: A method of comparing structural similarity of a first protein to a second protein that each have a fluorescence emission property, the method comprising:
a) providing a first solution comprising a component at a first concentration and the first protein;
b) altering the first concentration of the component in the first solution to a second concentration to form a second solution, wherein the second concentration changes the fluorescence emission property of the first protein;
c) measuring the fluorescence emission property of the first protein in the second solution;
d) comparing the fluorescence emission property of the first protein in the second solution to the fluorescence emission property of the second protein in a third solution comprising the component at the second concentration, wherein the second protein is a biosimilar of the first protein.

21: A method of comparing structural similarity of a first protein to a second protein that each have a fluorescence emission property, the method comprising:
a) providing a first solution comprising a component at a first concentration and the first protein;
b) altering the first concentration of the component to a second concentration to form a second solution, wherein the second concentration changes the fluorescence emission property of the first protein;
c) measuring the change in the fluorescence emission property of the first protein in the first solution and second solution;
d) providing a third solution comprising the component at the first concentration and the second protein, wherein the second protein is a biosimilar of the first protein;
e) altering the concentration of the component of the third solution to the second concentration to form a fourth solution;
f) measuring a change in the fluorescence emission property of the second protein in the third solution and fourth solution;
g) comparing the change in c) and the change in f).

22: The method of claim 20 or claim 21, wherein the first protein and second protein are manufactured by different processes.

23: The method of claim 22, wherein the first protein and second protein are manufactured using different host cells.

24: The method of claim 22, wherein the first protein and second protein are manufactured using different fermentation processes.

25: The method of claim 22, wherein the first protein and second protein are manufactured using different purification processes.

26: The method according to claim 20 or claim 21, wherein the component comprises one or more osmolytes.

27: The method according to claim 26, wherein the one or more osmolytes are selected from the group consisting of a glycerol, a polyethylene glycol, a buffer, a salt, urea, a non-ionic detergent, an ionic detergent, a non-ionic surfactant, an ionic surfactant, an acid, a hydrophobic molecule, a natural osmolyte, and combinations thereof.

28: The method according to claim 26, wherein the osmolyte is acetate buffer.

29: The method according to claim 27, wherein the natural osmolyte is selected from the group consisting of trimethylamine N-oxide (TMAO), dimethylsulfoniopropionate, trimethylglycine, sarcosine, betaine, glycerophosphorylcholine, myoinositol, and taurine.

30: The method according to claim 20 or 21, wherein the component comprises polysorbate.

31: The method according to claim 20 or 21, wherein the fluorescence emission property is fluorescence emission wavelength.

32: The method according to claim 20 or 21, wherein the fluorescence emission property is fluorescence emission intensity.

33: The method according to claim 20 or 21, wherein the fluorescence emission property is measured using an excitation wavelength between 150 and 300 nm.

34: The method according to claim 31, wherein the excitation wavelength is 257 nm.

35: The method according to claim 31, wherein the excitation wavelength is 274 nm.

36: The method according to claim 31, wherein the excitation wavelength is 280 nm.

37: The method according to claim 20 or 21, wherein the first protein and second protein each comprise a fluorescent active amino acid residue selected from the group consisting of tyrosine, tryptophan, and phenylalanine.

38: The method according to claim 20 or 21, wherein the first protein and second protein are selected from the group consisting of: antibodies, antibody fragments, vaccines, therapeutic proteins, enzymes, protein digests, and denatured proteins.

39: The method according to claim 37, wherein the first protein and second protein are therapeutic proteins.

40: The method according to claim 37, wherein the first protein and second protein are antibodies.
